(19) United States
(12) Patent Application Publication
(10) Pub. No.: US 2002/0120321 A1
(43) Pub. Date: Aug. 29, 2002

(54) Stent retention mechanism

(76) Inventors: Richard C. Gunderson, Maple Grove, MN (US); Daniel J. Vreeman, Rogers, MN (US); Bryan A. Claseman, Maple Grove, MN (US)

Correspondence Address: MERCHANT & GOULD PC, P.O. BOX 2903, MINNEAPOLIS, MN, 55402-0903, US

(21) Appl. No.: 09/793,668

(22) Filed: Feb. 26, 2001

(51) Int. Cl. A61F 2/06

(52) U.S. Cl. 623/1.11

(57) ABSTRACT

A stent delivery system for an intraluminal stent includes an elongated flexible member having a distal end and a proximal end. An expandable balloon is disposed on the distal end. A stent is disposed surrounding the balloon. A protruding retention member is provided on the balloon for restraining the stent from axial movement relative to the balloon.

### I. BACKGROUND OF THE INVENTION

1. Field of the Invention

This invention pertains to a system for delivering a stent to a site in a body lumen. More particularly, this invention pertains to a stent delivery system with improved structure for retaining a stent on a balloon.

2. Description of Prior Art

Stents are widely used for supporting a lumen structure in a patient's body. For example, stents may be used to maintain patency of a coronary artery , other blood vessel or other body lumen.

Stents are generally tubular structures formed of metal or other materials (e.g., plastic). Stents are passed through the lumen in a collapsed state. At the point of an obstruction or other deployment site in the lumen, the stent is expanded to an expanded diameter to support the lumen at the deployment site.

Some stents are balloon expandable stents. Such stents are carried through the lumen in a reduced diameter over a collapsed balloon at a distal tip of a catheter. At the deployment site, the balloon is inflated. Inflation of the balloon exerts a radial force against an inner cylindrical wall of the stent. The radial force causes the stent to expand to its expanded diameter supporting the lumen. Following full expansion of the stent, the balloon is collapsed such that the balloon and catheter can be withdrawn from the stent within the lumen thereby leaving the stent in place supporting the vessel.

From time to time, a stent may slip from a balloon such that the stent moves axially relative to the balloon and the catheter. Such event is undesirable and can adversely affect desired positioning of the stent. Commonly assigned U.S. patent application No. 09/404,418 provides one mechanism for addressing retention of a stent on a balloon. That application teaches providing the interior surface of the stent with a roughened surface such that there is enhanced friction between the balloon and the stent reducing the likelihood of relative axial movement or slippage between the stent and the balloon.

It is an object of the present invention to provide further structure for reducing the likelihood of relative axial movement or slippage between a stent and a balloon.

### II. SUMMARY OF THE INVENTION

According to a preferred embodiment of the present invention, a stent delivery system is disclosed for placement of an intraluminal stent in a body lumen. The stent is expandable from a reduced first diameter to an expanded second diameter by application of a radial force to an interior of the stent. The stent delivery system includes an elongated flexible member having a distal end and a proximal end. The flexible member has a member lumen extending throughout the entire axis of the flexible member from the distal end through the proximal end. An expandable balloon is disposed on the distal end with the balloon in fluid flow communication with the member lumen. A fluid port is provided at the proximal end in communication with the member lumen. A stent having a reduced first diameter is disposed surrounding the balloon with the balloon in a collapsed state. A protruding retention member is provided on the balloon for restraining the stent from axial movement relative to the balloon.

### III. BRIEF DESCRIPTION OF THE DRAWINGS

FIG. 1 is a side elevation view of an expandable balloon in an expanded state with a stent carried on the balloon and with the stent retained in place by a retention member according to the present invention;

FIG. 2 is a side longitudinal sectional view of the stent delivery system of FIG. 1;

FIG. 3 is an end view of an alternative embodiment of the present invention showing a balloon only partially inflated and without showing a stent for ease of illustration;

FIG. 4 is a view of FIG. 3 showing the balloon still further deflated and with folds of the balloon wrapped in a spiral manner around an axis of the stent delivery system;

FIG. 5 is a side elevation view of a still alternative embodiment of the present invention; and

FIG. 6 is an end view of the embodiment of FIG. 5.

### IV. DESCRIPTION OF THE PREFERRED EMBODIMENT

With reference now to the various drawing figures in which identical elements are numbered identically throughout, a description of a preferred embodiment of the present invention will now be provided. The present invention will be described with reference to a balloon carried on a so-called coaxial catheter. Coaxial catheters contain two catheters with an inner catheter concentrically placed within an outer catheter. The spacing between the inner and outer catheter defines a fluid lumen for passage of a fluid from a proximal end to the interior of a balloon at a distal end of the catheters. In a coaxial catheter, the balloon is connected to both the outer and inner catheters. It will be appreciated that while the present invention will be described with reference to coaxial catheters, the present invention is applicable to any other balloon catheter technology including stent delivery systems having a single catheter with multiple lumens, or rapid exchange catheters.

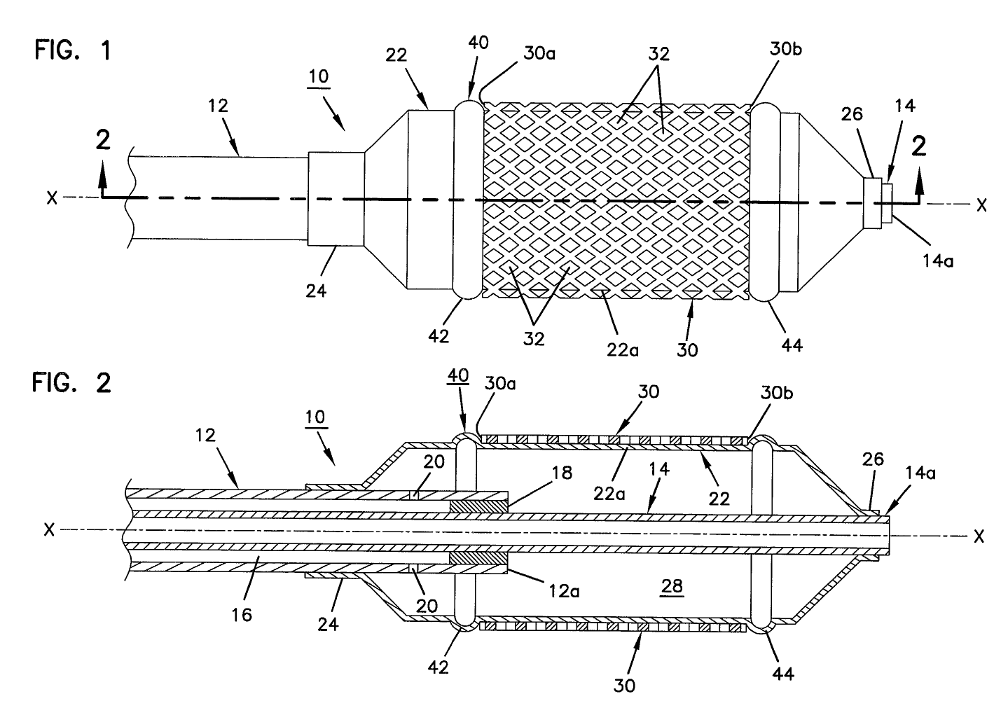

With initial reference to FIGS. 1 and 2, the stent delivery system 10 is shown in conjunction with a coaxial catheter having an outer catheter 12 and an inner catheter 14. A distal end 14a of the inner catheter extends beyond a distal end 12a of the outer catheter 12.

The inner catheter 14 is hollow for the stent delivery system 10 to be advanced over a pre-positioned guide wire (not shown). Both catheters 12, 14 terminate at a proximal end (not shown) exterior of the body. Opposing surfaces of the catheters 12, 14 define an annular lumen 16 extend along the length of the delivery system 10. The proximal ends of the catheters 12, 14 extend out of the body and include a port for delivery of fluid into the annular lumen 16 or withdrawal of fluid from the annular lumen 16 as may be desired by an operator. It will be appreciated that such ports and catheters thus described are well known in the prior art and examples of such are shown in U.S. Pat. No. 5,759,191 incorporated herein by reference.

The distal end 12a of the outer catheter 12 is bonded to the inner catheter 14 by a spacing ring 18. Ports 20 are formed through the wall of the outer catheter 12 at the distal end 12a.

A balloon 22 is provided at the distal end 12a of the outer catheter 12. The balloon 22 surrounds the distal end 12a and includes a proximal neck down portion 24 that is bonded to the outer surface of the outer catheter 12. The balloon 22 has a distal neck down portion 26 is bonded to the outer surface of the inner catheter 14 adjacent to distal end 14a.

The neck down portions 24, 26 being sealed to the catheters 12, 14 results in the balloon 22 having a sealed interior 28 which surrounds and communicates with the ports 20. Accordingly, fluid can be passed through the lumen 16 and ejected through the ports 20 and into the volume 28 for the purpose of inflating the balloon 22. Also, fluid can be evacuated from the volume 28 through ports 20 resulting in deflation of the balloon 22.

In FIGS. 1 and 2, a stent 30 is schematically shown surrounding the balloon and being carried on the balloon 22. Stent 30 is only schematically shown and may be any balloon expandable stent. Such stents 30 commonly include an open cell construction such that the stent 30 has a polarity of open cells 32 formed completely through the side cylindrical wall of the stent 30. An exemplary stent is shown in U.S. patent application Ser. No. 09/765,725 filed on Jan. 18, 2001 and entitled STENT, which is hereby incorporated by reference.

The stent 30 is cylindrical and is mounted with its cylindrical axis being coaxial with the longitudinal axis X−X of the catheters 12, 14. The stent 30 is compressed to its reduced diameter state on a deflated balloon 22 with the reduced size structure being passed through a lumen to an occluded site in a vessel or other body lumen. At the site, the balloon 22 may be inflated by injecting an inflation media (such as a contrast media with or without saline solution) into the lumen 16 and through ports 20 into the balloon interior 28.

The expansion of the balloon 22 results in a radial force being applied against the interior cylindrical surface of the stent 30 causing the stent 30 to expand. A plurality of open cells 32 permit such expansion as well as provide longitudinal flexibility to the stent 30. After the stent 30 is expanded, the balloon 22 is deflated and withdrawn from the expanded stent leaving the expanded stent 30 in the body lumen.

It will be appreciated the structure thus described is well known in the prior art and forms no part of this invention per se. Instead, the present invention is directed toward a novel mechanism for preventing slippage of the stent 30 on the balloon 22.

In the embodiment of FIGS. 1 and 2, the balloon 22 is provided with protruding retention member 40 in the form of two spaced apart radial rings 42, 44 on the cylindrical surface of the balloon 22. The rings 42, 44 are spaced apart approximate to an axial length of the stent 30. The rings 42, 44 have a diameter greater than a diameter of a cylindrical portion 22a of the expanded balloon 22.

The stent 30 is mounted surrounding the cylindrical portion 22a. Therefore, axial ends 30a, 30b of the stent 30 oppose the rings 42, 44 with the rings 42, 44 blocking axial movement of the stent 30 on the balloon 22. Preferably, the rings 42, 44 block axial movement of the stent 30 on the balloon 22 during expansion of the balloon, as well as during transport of the stent through a patient's vasculature prior to expansion.

In the embodiment of FIGS. 1 and 2, the rings 42, 44 are formed completely surrounding the circumference of the balloon 22 and are shown as being integrally molded with the material of the balloon 22. It will be appreciated that the present invention can be used without the need for molding the rings 42, 44 with the balloon 22. Instead, the rings 42, 44 could be any bio-compatible, flexible material adhered or otherwise bonded to the external surface of the cylindrical portion 22a of the balloon 22. The rings 42, 44 can also be incorporated into the cylindrical portion 22a of the balloon 22.

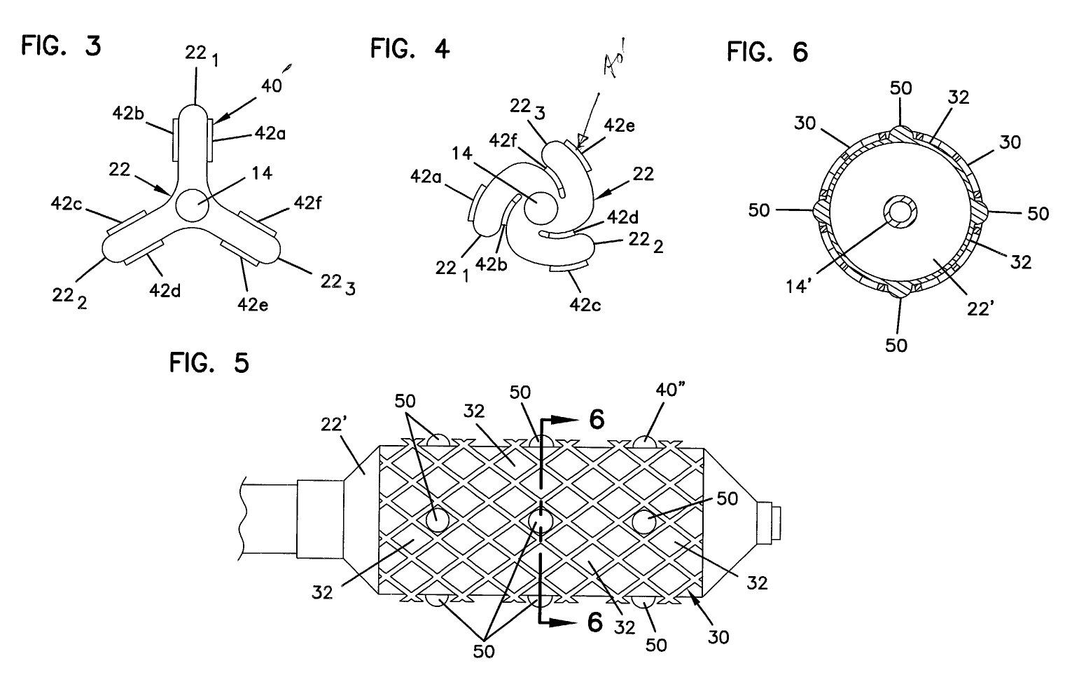

The rings 42, 44 need not be continuous rings. For example, continuous rings 42, 44 may interfere with folding of the balloon 22. This is illustrated in FIGS. 3 and 4. FIG. 3 shows so called tri-fold balloon 22 where a partially inflated balloon presents three folds 22₁, 22₂, 22₃ around central catheter 14. The folds 22₁, 22₂, 22₃ are then spiral wound around the catheter 14 as illustrated in FIG. 4 to provide the most compact shape for the collapsed state balloon 22.

Continuous rings 42, 44 could interfere with the folding of the balloon. As a result, and as shown in FIGS. 3 and 4, the protrusion member 40 is not shown as continuous rings but are shown as a segmented ring 40′ illustrated as being a polarity of ring segments 42a-42f to rest on opposite sides of the folds 221, 222, 223 and not at the apex of the folds 22₁, 22₂, 22₃ or at the valleys of the folds 22₁, 22₂, 22₃ and thereby avoid interference with the folding of the balloon 22.

FIGS. 5 and 6 illustrate a still further embodiment of the present invention. In FIGS. 5 and 6 the protrusion member 40″ is not rings at opposite ends of the stent 30. Instead, the protrusion member 40″ is a plurality of individual protrusions 50 formed along the cylindrical wall 22a of the balloon 22′. The individual protrusions 50 are positioned to project through the cells 32 of the stent 30 and thereby prevent axial slippage between the stent 30 and the balloon 22′. Protrusion member 40″ may be of any shape or configuration and will preferably be complimentarily shaped to the geometry of the cells 32 to mate with the stent design.

As a result of the foregoing, the stent is mechanically secured to a balloon. It has been shown how the objects of the invention have been attained in a preferred manner. Modifications and equivalents of the disclosed concepts are intended to be included within the scope of the claims which are appended hereto.

What is claimed is:

1. A stent delivery system for placement of an intraluminal stent in a body lumen wherein said stent is expandable from a reduced first diameter to an enlarged second diameter by application of a radial force to an interior of said stent, said stent delivery system comprising:
an elongated flexible member having a distal end and a proximal end and having a member lumen extending between said distal and proximal ends;
an expandable balloon exposed adjacent said distal end and in fluid flow communication with said member lumen;
a fluid port at said distal end and in fluid flow communication with said member lumen;
a stent disposed on said balloon and surrounding said balloon; and
a protruding retention member on said balloon for restraining said stent from axial movement relative to said balloon.

2. A stent delivery system according to claim 1 wherein said stent has a predetermined axial length, said retention member including a protrusion on said balloon opposing at least one end of said stent.

3. A stent delivery system according to claim 2 wherein said retention member includes first and second protrusions spaced apart greater than said axial length of said stent and with said stent disposed between said first and second protrusions.

4. A stent delivery system according to claim 2 wherein said protrusion circumscribe said balloon.

5. A stent delivery system according to claim 4 when said protrusion is continuous.

6. A stent delivery system according to claim 4 when said protrusion is segmented into a parallelity of individual members.

7. A stent delivery system according to claim 1 wherein said stent includes an open cell, said protrusion member disposed to project at least partially through said open cell.

8. A stent delivery system according to claim 7 wherein said stent includes a plurality of open cells and further comprising a plurality of protrusion members each protruding into individual ones of said open cells.

9. A stent delivery system according to claim 7 wherein said protrusion member is shaped generally complementarily to a geometry of said open cell.
